(19) United States
(12) Patent Application Publication
(10) Pub. No.: US 2007/0100291 A1
(43) Pub. Date: May 3, 2007

(54) Safety syringe

(76) Inventors: Chin-Hsia Huang, Taichung Hsien (TW); Fen-Fang Chang, Taichung Hsien (TW)

Correspondence Address: DLA PIPER RUDNICK GRAY CARY US, LLP, 2000 UNIVERSITY AVENUE, E. PALO ALTO, CA, 94303-2248, US

(21) Appl. No.: 11/590,426

(22) Filed: Oct. 30, 2006

(30) Foreign Application Priority Data
Oct. 31, 2005 (TW) 094138109

(51) Int. Cl. A61M 5/32; A61M 5/00

(52) U.S. Cl. 604/198; 604/195; 604/110; 604/192; 604/263

(57) ABSTRACT

A safety syringe includes a barrel, a needle hub, a plunger, an elastic piston, and a pushing member. The barrel has a shoulder. The needle hub extends from the shoulder of the barrel, and is provided with a first engaging member. The plunger extends into the barrel, and is provided with a second engaging member. The piston is sleeved on the plunger, is disposed rearward of the second engaging member, is received in and is in frictional contact with the barrel, and has a hollow body, and a restricting member that extends radially and inwardly from the hollow body. The pushing member extends radially from the plunger. The plunger is movable together with the piston relative to the barrel to a first position, and is movable relative to the piston from the first position to a second position.

### CROSS-REFERENCE TO RELATED APPLICATION

This application claims priority of Taiwanese application no. 094138109, filed on Oct. 31, 2005.

### BACKGROUND OF THE INVENTION

1. Field of the Invention

This invention relates to a syringe, more particularly to a safety syringe that can be safely disposed.

2. Description of the Related Art

FIG. 1 illustrates a conventional safety syringe that includes a barrel 50, a needle hub 60, a hub connector 61, a plunger 70, an elastic piston 80, and a pushing member 90. The barrel 50 has a medicine-storing portion 501, a needle-connecting portion 502 that is reduced in cross-section from the medicine-storing portion 501, and a shoulder 503 formed between the medicine-storing portion 501 and the needle-connecting portion 502. The hub connector 61 is mounted movably in the barrel 50, and has front and rear end portions 611, 612. The rear end portion 612 of the hub connector 61 abuts against the shoulder 503 of the barrel 50 and is provided with a first engaging member 610 therein. The needle hub 60 engages threadedly the front end portion 611 of the hub connector 61. A needle 100 is coupled to a front end of the needle hub 60. The plunger 70 extends into the medicine-storing portion 501 of the barrel 50, and has a front end that is provided with a second engaging member 701. The piston 80 is sleeved on the plunger 70, is disposed rearward of the second engaging member 701, is received in and is in frictional contact with the medicine-storing portion 501 of the barrel 50, and has front and rear ends 801, 802. The pushing member 90 extends radially from the plunger 70, and abuts against the rear end 801 of the piston 80. With further reference to FIG. 2, the plunger 70 is movable relative to the barrel 50 in a frontward direction from a first position, in which the first and second engaging members 611, 701 are spaced apart from each other, to a second position, in which the first and second engaging members 611, 701 engage each other. At this time, the hub connector 61 together with the needle hub 60 and the needle 100 may be drawn into the medicine-storing portion 501 of the barrel 50 by pulling the plunger 70.

The aforementioned conventional safety syringe is disadvantageous in that, when the plunger 70 is moved to the first position, a gap exists between the front end 801 of the piston 80 and the rear end portion 612 of the hub connector 61. As such, the conventional safety syringe still has medicine fluid residue left in the gap after use. Moreover, the gap contains trapped air, which decreases suction force of the conventional safety syringe when the plunger 70 is pulled. Further, the plunger 70 is easily moved to the second position when accidentally pushed.

### SUMMARY OF THE INVENTION

Therefore, the object of the present invention is to provide a safety syringe that can overcome the aforesaid drawbacks of the prior art.

According to the present invention, a safety syringe comprises a barrel, a needle hub, a plunger, an elastic piston, and a pushing member. The barrel has a medicine-storing portion, a needle-connecting portion that is reduced in cross-section from the medicine-storing portion, and a shoulder that is formed between the medicine-storing portion and the needle-connecting portion. The needle hub extends into the needle-connecting portion of the barrel, and is provided with a first engaging member therein. The plunger extends into the medicine-storing portion of the barrel, and has a front end that is provided with a second engaging member. The elastic piston is sleeved on the plunger, is disposed rearward of the second engaging member, is received in and is in frictional contact with the medicine-storing portion of the barrel, and has a hollow body that has a rear end, and a restricting member that extends radially and inwardly from the rear end of the hollow body. The pushing member extends radially from the plunger. The plunger is movable together with the piston relative to the barrel to a first position, in which the pushing member is disposed rearwardly of and abuts against the restricting member, in which the piston abuts against the shoulder of the barrel, and in which the first engaging member is spaced apart from the second engaging member. The plunger is movable relative to the piston in a frontward direction from the first position to a second position, in which the piston abuts against the shoulder of the barrel and in which the first and second engaging members engage each other.

### BRIEF DESCRIPTION OF THE DRAWINGS

Other features and advantages of the present invention will become apparent in the following detailed description of the preferred embodiment with reference to the accompanying drawings, of which:

FIG. 1 is a fragmentary partly sectional view of a conventional safety syringe when a plunger thereof is moved to a first position;

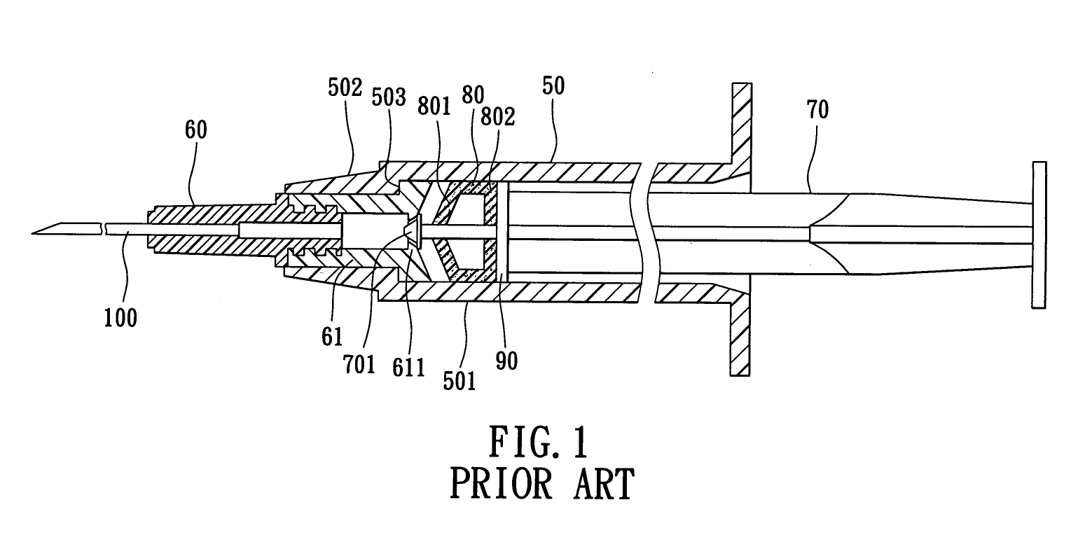

FIG. 2 is a fragmentary partly sectional view of the conventional safety syringe when the plunger thereof is moved to a second position;

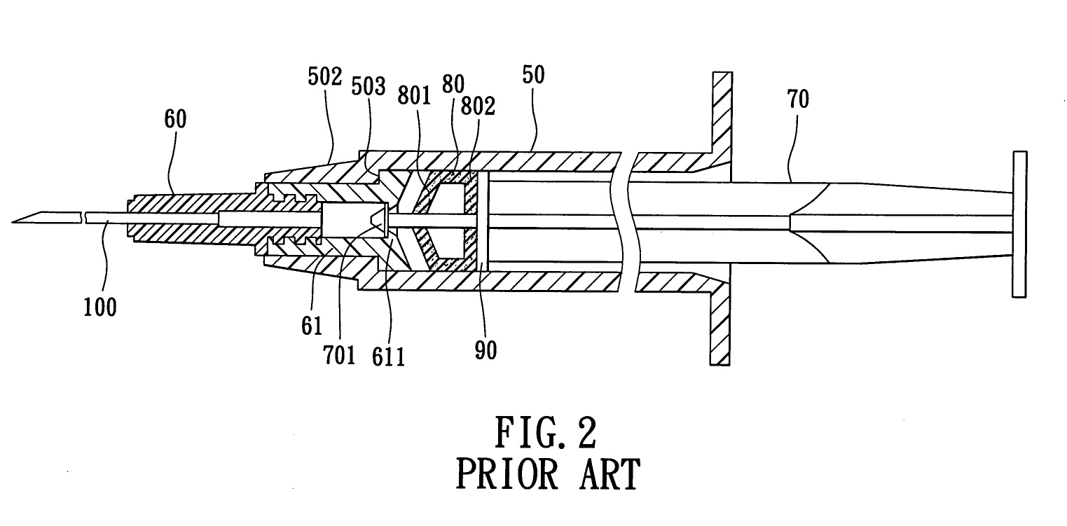

FIG. 3 is an exploded fragmentary partly sectional view of the preferred embodiment of a safety syringe according to this invention;

FIG. 4 is a fragmentary partly sectional view of the preferred embodiment when a plunger thereof is disposed at an initial position;

FIG. 5 is a fragmentary partly sectional view of the preferred embodiment when the plunger thereof is moved to a first position;

FIG. 6 is a fragmentary partly sectional view of the preferred embodiment when the plunger thereof is moved to a second position;

FIG. 7 is a fragmentary partly sectional view of the preferred embodiment when the plunger thereof is moved to a third position;

FIG. 8 is a fragmentary partly sectional view to illustrate a guiding piece of the preferred embodiment; and

FIG. 9 is a fragmentary partly sectional view to illustrate a plunger being guided by the guiding piece of the preferred embodiment.

### DETAILED DESCRIPTION OF THE PREFERRED EMBODIMENT

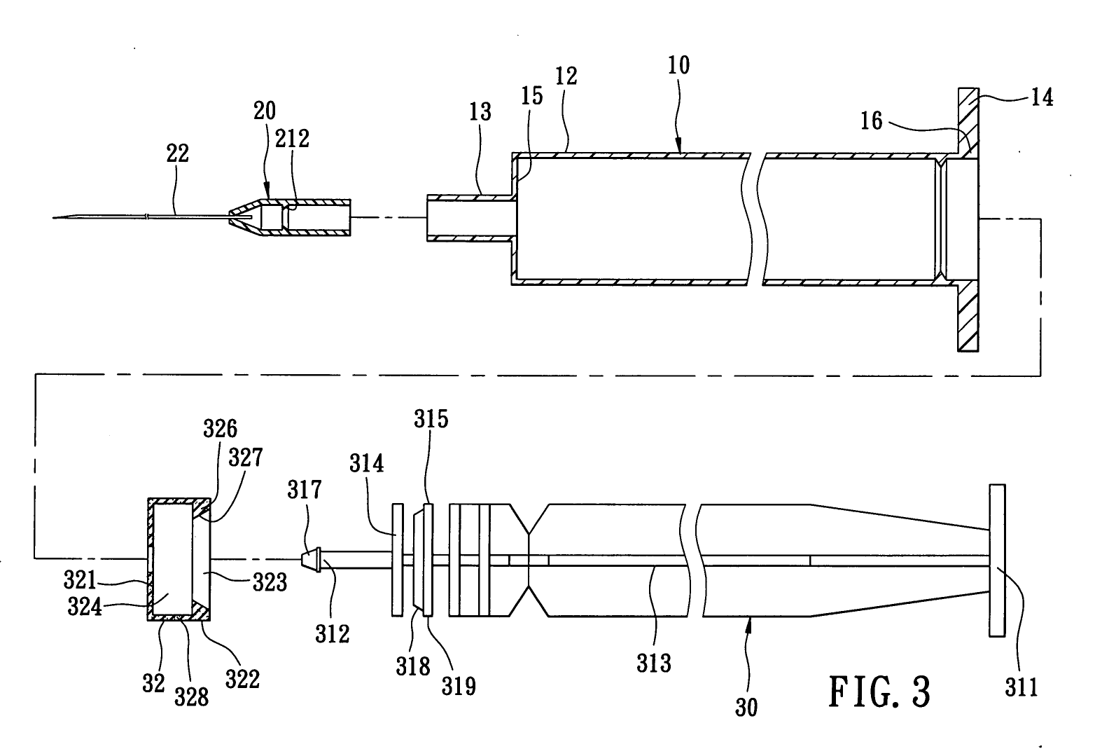

Referring to FIG. 3, the preferred embodiment of a safety syringe according to this invention is shown to include a barrel 10, a needle hub 20, a plunger 30, an elastic piston 32, and first and second pushing members 315, 314.

The barrel 10 has a medicine-storing portion 12 and a needle-connecting portion 13. The medicine-storing portion 12 of the barrel 10 has opposite front and rear ends 15, 16. The needle-connecting portion 13 of the barrel 10 is reduced in cross-section from the medicine-storing portion 12. As such, a shoulder, which serves as the front end 15 of the barrel 10, is formed between the medicine-storing portion 12 and the needle-connecting portion 13 of the barrel10. The barrel 10 is provided with a finger flange 14 that extends radially and outwardly from the rear end 16 of the medicine-storing portion 12 of the barrel 10.

The needle hub 20 extends into the needle-connecting portion 13 of the barrel 10, and is provided with a first engaging member 212 therein. In this embodiment, the first engaging member 212 is an annular tapered flange.

The safety syringe further includes a hypodermic needle 22 that extends from the needle hub 20.

The plunger 30 extends into the medicine-storing portion 12 of the barrel 10, and has opposite front and rear ends 312, 311, and a middle portion 313 that interconnects the front and rear ends 312, 311 of the plunger 30. The rear end 311 of the plunger 30 is disposed externally of the barrel 10. The front end 312 of the plunger 30 is disposed in the barrel 10, and is provided with a second engaging member 317. In this embodiment, the second engaging member 317 is an annular projection.

The piston 32, preferably made from a plastic material, is disposed rearward of the second engaging member 317, is sleeved on the middle portion 313 of the plunger 30, and is received in and is in frictional contact with the medicine-storing portion 12 of the barrel 10. In this embodiment, the piston 32 has a hollow body 328 and a restricting member 326. The hollow body 328 of the piston 32 has a front end 321, and a rear end 322 that is opposite to the front end 321 of the hollow body 328. The restricting member 326 extends radially and inwardly from the rear end 322 of the hollow body 328, cooperates with the hollow body 328 to define an inner space 324 therebetween, and defines a rear opening 323 that is in spatial communication with the inner space 324 and that is defined by a tapered wall 327.

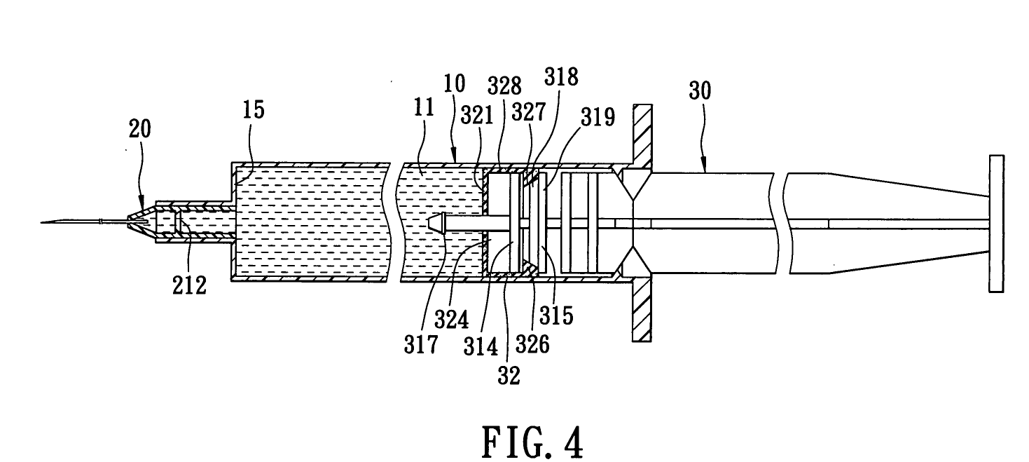

The first pushing member 315 is disposed rearward of the second engaging member 317, and extends radially from the middle portion 313 of the plunger 30. In this embodiment, the first pushing member 315 is disposed externally of the piston 32 (see FIG. 4), and has a tapered front end portion 318 and an annular rear end portion 319.

The second pushing member 314 is disposed between the second engaging member 317 and the first pushing member 315, and extends radially from the middle portion 313 of the plunger 30. In this embodiment, the second pushing member 314 is received in the inner space 324 and is in frictional contact with the piston 32 (see FIG. 4).

Referring to FIG. 4, at an initial position in which the medicine-storing portion 12 of the barrel 10 already contains medicine fluid 11, the piston 32 is disposed at a distance from the shoulder 15 of the barrel 10. The tapered front end portion 318 of the first pushing member 315 extends into the rear opening 323 (see FIG. 3) and is in frictional contact with the tapered wall 327 of the restricting member 326 of the piston 32. The annular rear end portion 319 of the first pushing member 315 abuts against the restricting member 326. The second pushing member 314 is disposed in the inner space 324. The first engaging member 212 and the second engaging member 317 are spaced apart from each other.

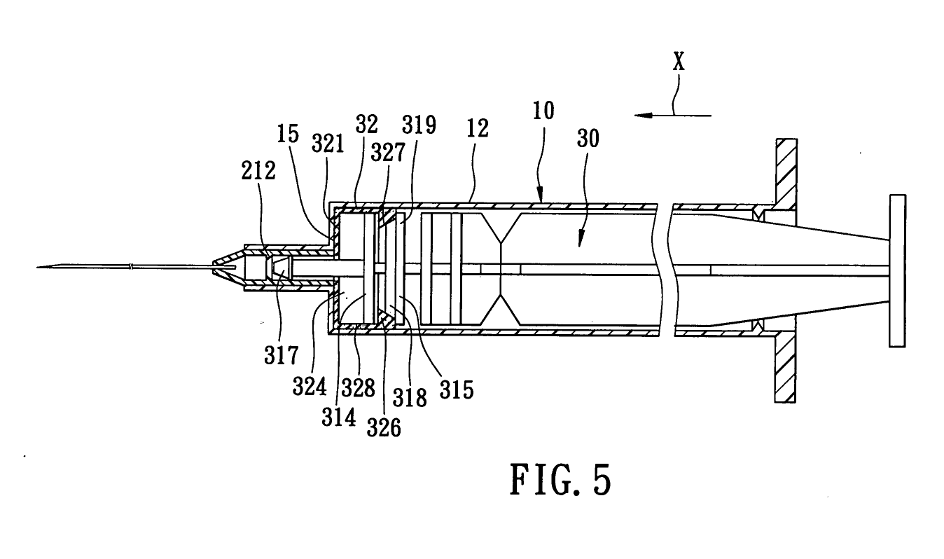

Referring to FIG. 5, the plunger 30 is movable together with the piston 32 relative to the barrel 10 in a frontward direction, as indicated by arrow (X), from the initial position to a first position, in which the medicine fluid 11 (see FIG. 4) is discharged from the medicine-storing portion 12 of the barrel 10, in which the front end 321 of the hollow body 328 of the piston 32 abuts against the shoulder 15 of the barrel 10, in which the annular rear end portion 319 of the first pushing member 315 is disposed rearwardly of and abuts against the restricting member 326, and in which the first and second engaging members 212, 317 are spaced apart from each other. The tapered front end portion 318 of the first pushing member 315 is still disposed in the rear opening 323 (see FIG. 3) and is in frictional contact with the tapered wall 327 of the restricting member 326 of the piston 32. The second pushing member 314 is still disposed in the inner space 324. It is noted that, when the plunger 30 is at this position, there is no gap existing between the front end 321 of the hollow body 328 of the piston 32 and the shoulder 15 of the barrel 10. As such, there is no medicine fluid residue in the medicine-storing portion 12 of the barrel 10. Moreover, since no gap exists between the front end 321 of the hollow body 328 of the piston 32 and the shoulder 15 of the barrel 10, there is no trapped air in the medicine-storing portion 12 of the barrel 10. As such, suction force of the safety syringe of this invention is enhanced when the plunger 30 is pulled. Further, since the medicine fluid may be fully discharged from the medicine-storing portion 12 of the barrel 10 without resulting in the engagement between the first and second engaging members 212, 317, the safety syringe of this invention is reusable.

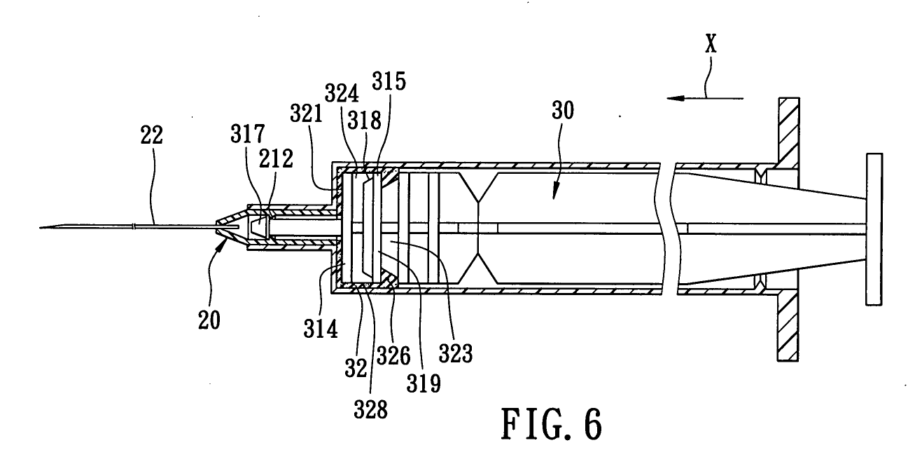

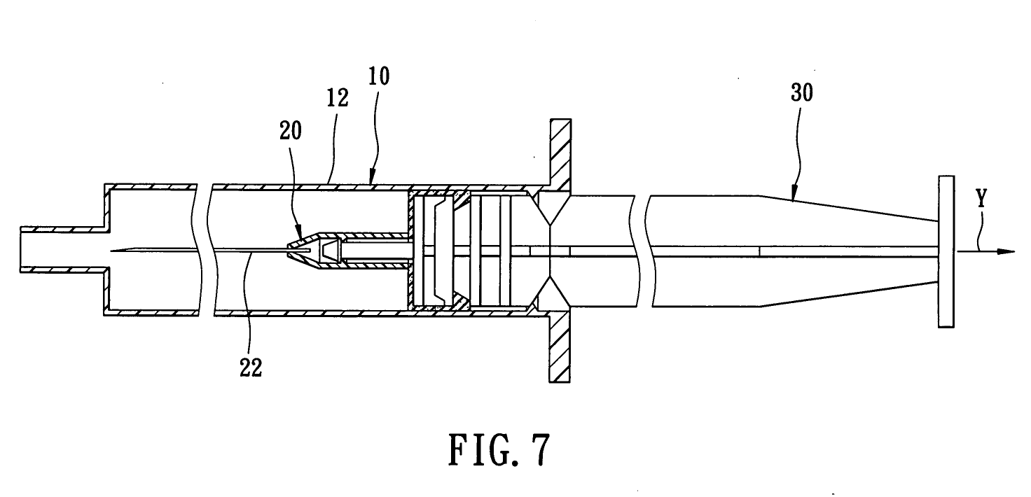

Referring to FIG. 6, due to abutment between the front end 321 of the hollow body 328 of the piston 32 and the shoulder 15 of the barrel 10, the plunger 30 is movable relative to the piston 32 in the frontward direction (X) from the first position to a second position, in which the tapered front end portion 318 and the non-tapered rear end portion 319 of the first pushing member 315 are received in the inner space 324 through the rear opening 323 in the restricting member 326 of the piston 32, in which the second pushing member 314 is still disposed in the inner space 324 and abuts against the front end 321 of the hollow body 328 of the piston 32, and in which the first engaging member 212 and the second engaging member 317 engage each other. At this time, the needle hub 20 and the hypodermic needle 22 may be retracted into the medicine-storing portion 12 of the barrel 10 by moving the plunger 30 relative to the barrel 10 in a rearward direction, as indicated by arrow (Y), opposite to the frontward direction (X), as best shown in FIG. 7, from the second position to a third position.

In this embodiment, when the plunger 30 is at the initial position (see FIG. 4), a gap exists between the second pushing member 314 and the front end 321 of the hollow body 328 of the piston 32. When the plunger 30 is moved to the first position (see FIG. 5), the gap still exists between the second pushing member 314 and the front end 321 of the hollow body 328 of the piston 32. When the plunger 30 is moved to the second position (see FIG. 6), there is no gap between the second pushing member 314 and the front end 321 of the hollow body 328 of the piston 32. In an alternative embodiment, when the plunger 30 is moved to the second position, the gap between the second pushing member 314 and the front end 321 of the hollow body 328 of the piston 32 is simply reduced.

It is noted herein that it requires a certain amount of force before the first pushing member 315 can be pushed into the inner space 324 through the rear opening 323 in the restricting member 326 of the piston 32. As such, accidental movement of the plunger 30 to the second position can be deterred.

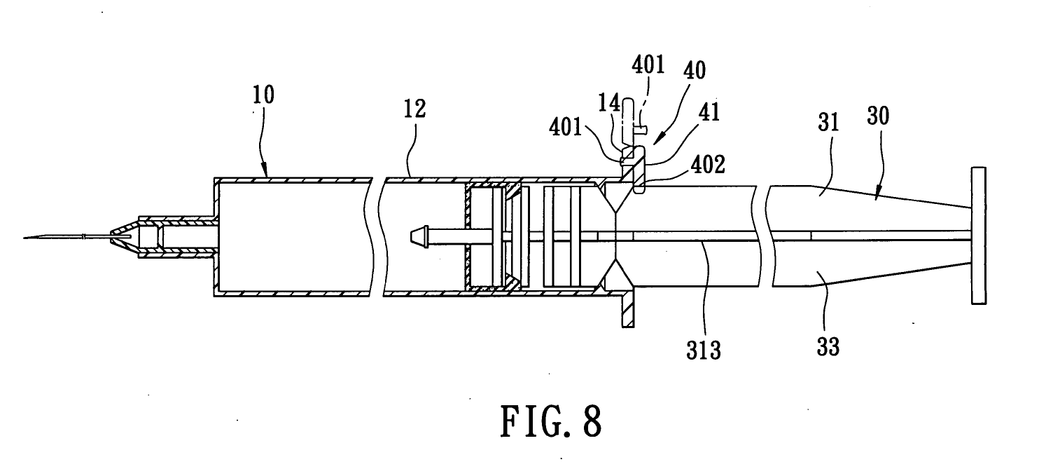

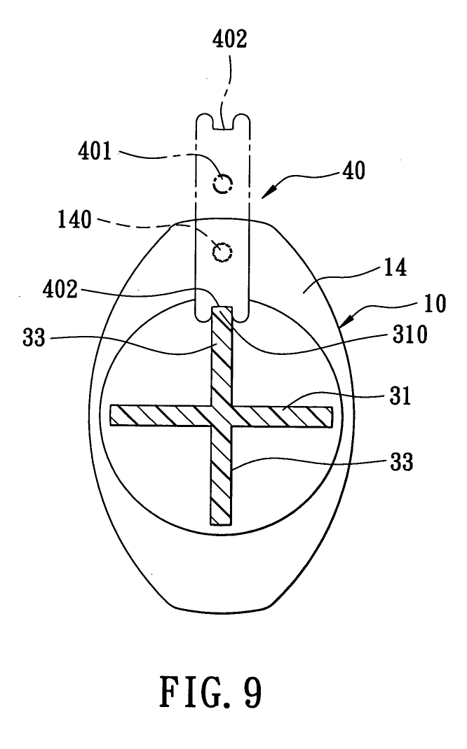

With further reference to FIGS. 8 and 9, the safety syringe further includes a guiding piece 40 for guiding movement of the plunger 30 in the medicine-storing portion 12 of the barrel 10. In particular, the middle portion 313 of the plunger 30 is provided with a guide member 31 that has a cross-shaped cross-section, and that includes a pair of crossed pieces 33. The finger flange 14 of the barrel 10 is formed with an engaging hole 140. The guiding piece 40 is provided with an engaging protrusion 401 that engages the engaging hole 140 in the finger flange 14 of the barrel 10, as best shown in FIG. 8, and is formed with a guiding groove 402 that receives slidably an edge 310 of one of the crossed pieces 33 of the plunger 30, as best shown in FIG. 8.

With the formation of the inner space 324 in the piston 32 and the restricting member 326 on the rear end 322 of the hollow body 328 of the piston 32, the aforesaid drawbacks associated with the prior art can be eliminated.

While the present invention has been described in connection with what is considered the most practical and preferred embodiment, it is understood that this invention is not limited to the disclosed embodiment but is intended to cover various arrangements included within the spirit and scope of the broadest interpretation so as to encompass all such modifications and equivalent arrangements.

What is claimed is:

1. A safety syringe, comprising:
a barrel having a medicine-storing portion, a needle-connecting portion that is reduced in cross-section from said medicine-storing portion, and a shoulder formed between said medicine-storing portion and said needle-connecting portion;
a needle hub extending into said needle-connecting portion of said barrel and provided with a first engaging member therein;
a plunger extending into said medicine-storing portion of said barrel, and having a front end that is provided with a second engaging member, and a rear end that is opposite to said front end of said plunger;
an elastic piston sleeved on said plunger, disposed rearward of said second engaging member, and received in and in frictional contact with said medicine-storing portion of said barrel, said piston having a hollow body that has a rear end, and a restricting member that extends radially and inwardly from said rear end of said hollow body; and
a first pushing member extending radially from said plunger;
wherein said plunger is movable together with said piston relative to said barrel to a first position, in which said first pushing member is disposed rearwardly of and abuts against said restricting member, in which said piston abuts against said shoulder of said barrel, and in which said first engaging member is spaced apart from said second engaging member; and
wherein said plunger is movable relative to said piston in a frontward direction from the first position to a second position, in which said piston abuts against said shoulder of said barrel and in which said first and second engaging members engage each other.

2. The safety syringe as claimed in claim 1, wherein said restricting member cooperates with said hollow body to define an inner space therebetween, and defines a rear opening that is in spatial communication with said inner space, said first pushing member is received in said inner space when said plunger is disposed at the second position.

3. The safety syringe as claimed in claim 2, further comprising a second pushing member extending radially from said plunger, received in said inner space, and in frictional contact with said piston.

4. The safety syringe as claimed in claim 2, wherein said rear opening in said restricting member of said piston is defined by a tapered wall.

5. The safety syringe as claimed in claim 4, wherein said first pushing member has a tapered front end portion and an annular rear end portion.

6. The safety syringe as claimed in claim 2, wherein said plunger further has a middle portion that interconnects said front and rear ends thereof, said middle portion of said plunger being provided with a guiding member that has a cross-shaped cross-section, and that includes a pair of crossed pieces, said barrel being provided with a finger flange that extends radially and outwardly from said rear end of said medicine-storing portion thereof, said safety syringe further comprising a guiding piece for guiding movement of said plunger in said barrel, said guiding piece engaging said finger flange of said barrel, and being formed with a guiding groove that receives slidably an edge of one of said crossed pieces of said guiding member therein.
